(19) United States
(12) Patent Application Publication
(10) Pub. No.: US 2008/0227112 A1
(43) Pub. Date: Sep. 18, 2008

(54) Global polyclonal antibodies, process for depleting commonly shared proteins by same, devices using same

(75) Inventor: Shu-Cai Huang, Madison, WI (US)

Correspondence Address: BRINKS HOFER GILSON & LIONE, P.O. BOX 10395, CHICAGO, IL, 60610, US

(73) Assignee: GeneTel Laboratories LLC

(21) Appl. No.: 12/075,335

(22) Filed: Mar. 11, 2008

Related U.S. Application Data
(60) Provisional application No. 60/906,186, filed on Mar. 12, 2007.

(51) Int. Cl. G01N 33/53; C07K 16/00; C12M 1/34

(52) U.S. Cl. 435/7.1; 530/389.1; 435/287.2

(57) ABSTRACT

A method for removing abundance proteins from a biological sample comprises passing the biological sample through a support. The support is coated with an avian polyclonal antibody. The avian polyclonal antibody is capable of binding to substantially all proteins in the biological sample with concentrations higher than a predetermined value. The method for removing abundance proteins from a biological sample further comprises collecting the pass-through fractions of the biological sample.

The present application claims priority to U.S. Provisional Patent Application Ser. No. 60/906,186, filed Mar. 12, 2007, the entirety of which is hereby incorporated by reference.

### FIELD OF THE INVENTION

The present disclosure relates to global polyclonal antibodies, a process for depleting commonly shared abundance proteins by same, and devices using same.

### BACKGROUND OF THE INVENTION

Biological samples contain proteins in a wide concentration range. For example, in human plasma, more than 10 orders of magnitude in concentration separate albumin from low abundance proteins. Moreover, the human plasma is estimated to contain about 500,000 protein forms and species. Yet only about 300 different proteins have been detected in the human plasma. Many functional and potentially disease-associated proteins remain to be identified. High abundance (mg/mL) and moderate abundance (μg/mL) proteins (collectively, “abundance proteins”) are estimated to account for over about 95% of total protein contents (in amount) and yet represent less than about 2% of protein species in the plasma. The rest of the about 98% protein species that are considered to be low abundance proteins (ng/mL or lower) only accounts for less than about 5% of total protein contents (in amount). Because of its complex nature, directly studying the plasma proteome by currently available proteomic technologies is difficult. For example, affinity column kits for depletion of multiple high abundance proteins have been developed by companies such as Agilient, GenWay Biotech, and Beckman Coulter. However, applications of these kits are limited in that only a few known abundance proteins can be removed.

Analysis of differential expression of proteins between cancerous and normal samples helps understand the neoplastic process and the mechanism of cancer drug action. Over the past decade, there have been improvements in technologies that enable the quantification and characterization of cancer-relevant proteins in cultured cancer cells, plasma and tumor tissue. For example, new protein markers that play a role in screening, monitoring and staging of some cancers have been discovered. Nevertheless, effective biomarkers are not currently available for most cancers and are generally nonexistent for early detection. Comparisons of proteomes between cancerous and normal samples will accelerate the progress in identifying cancer-relevant proteins. On the other hand, effective treatments are not currently available for most cancers either. Comparisons of low abundance proteomes from cancerous versus from normal samples such as plasma can result in identification of new biomarkers, and druggable proteins that can be useful in treatment.

Various tools and methods have been developed for proteomic analysis. These include two-dimensional polyacrylamide gel electrophoresis (2-D PAGE) analysis, matrix-assisted laser desorption/ionization time-of-flight mass spectrometry (MALDI-TOF MS), surface enhanced laser desorption ionization time-of-flight mass spectrometry (SELDI-TOF MS), isotope-coded affinity tag (ICAT), liquid chromatography mass spectrometry (LC-MS), tandem mass spectrometry (MS/MS), and the like. The sensitivity of these available technologies, however, is obscured by the presence of proteins that are both abundant and commonly shared in both normal and cancerous samples. The presence of these commonly shared abundance proteins limits the ability of these techniques to identify potential cancer-relevant proteins that are of low abundance in the biological samples.

To increase detection sensitivity, it is necessary to develop technologies or methods that are able to remove the obscuring, commonly shared abundance proteins from the biological samples. Several techniques have been developed for removing such commonly shared abundance proteins, including immunoaffinity (antibody) depletion. It is shown that immunoaffinity depletion of certain abundance proteins from biological samples can help increase the detection sensitivity of other proteomic tools, such as 2-D gel, MALDI-TOF MS and LC-MS. For example, immunoaffinity depletion columns manufactured by Agilient, GenWay Biotech, and Beckman Coulter were used to remove certain known abundance proteins, such as albumin, from plasma, using affinity columns. These methods, however, utilize specific antibodies coupled to resins, which are then used to deplete the known abundance proteins from the biological samples.

These above-discussed methods are aimed to remove one or several known abundance proteins from the biological samples to facilitate further studies by other proteomics tools. These methods are limited in that they are only effective in removing a few known abundance proteins from the biological samples. Many commonly shared abundance proteins, however, remain in the samples to be analyzed, which can interfere the identification of low-abundance, cancer-relevant proteins.

Therefore, it is desirable to develop a method to remove most, if not all, commonly shared abundance proteins from any total protein samples. The present disclosure discloses global polyclonal antibodies derived from animals such as chickens as a filter to deplete commonly shared proteins from the total protein samples in order to enrich disease-specific proteins for further proteomics analysis and discovery of protein markers.

### BRIEF SUMMARY OF THE INVENTION

In one aspect, a method for removing abundance proteins from a biological sample comprises passing the biological sample through a support. The support is coated with an avian polyclonal antibody. The avian polyclonal antibody is capable of binding to substantially all proteins in the biological sample with concentrations higher than a predetermined value. The method for removing abundance proteins from a biological sample further comprises collecting the pass-through fractions of the biological sample.

In another aspect, a method for enriching a protein associated with a certain disease comprises removing proteins from a disease biological sample using an avian polyclonal antibody against total proteins of a corresponding normal biological sample, and collecting the pass-through fractions of the disease biological sample.

In yet another aspect, a method for enriching a protein expressed in a normal tissue or cell but not in a corresponding disease tissue or cell comprises removing proteins from a normal biological sample using an avian polyclonal antibody against total proteins of a corresponding disease biological sample, and collecting the pass-through fractions of the normal biological sample.

In still another aspect, the present disclosure discloses an avian polyclonal antibody against abundance proteins in a biological sample. The avian polyclonal antibody is produced by immunizing an avian animal with the total proteins of a biological sample, and collecting the avian polyclonal antibody from the resultant animal. The avian polyclonal antibody is capable of binding to substantially all proteins in the biological sample with concentrations higher than a predetermined value.

In further another aspect, a device for removing proteins from a biological sample comprises a support, and an avian polyclonal antibody coated to the support. The avian polyclonal antibody is against abundance proteins in a biological sample. The avian polyclonal antibody is produced by immunizing an avian animal with the total proteins of a biological sample, and collecting the avian polyclonal antibody from the resultant animal. The avian polyclonal antibody is capable of binding to substantially all proteins in the biological sample with concentrations higher than a predetermined value.

### BRIEF DESCRIPTION OF THE DRAWINGS

FIG. 1 illustrates a method to deplete commonly shared proteins by global polyclonal antibody columns for enrichment of potential protein biomarkers associated with specific disease or biological function.

### DETAILED DESCRIPTION OF THE INVENTION

The present disclosure provides a novel method for depleting most, if not all, commonly shared proteins from biological samples, for example, between normal and disease total protein samples. Specifically, the present disclosure uses polyclonal antibodies generated to respond globally against all known and unknown commonly shared abundance proteins contained in both normal and disease samples to fabricate affinity columns. These columns then are used to deplete or remove the commonly shared abundance proteins from the total protein samples. This novel method allows rapid, easy, and efficient enrichment of disease-associated proteins for further proteomic analysis and for discovery of potential protein markers. The depleted samples obviate the interference caused by the shared, abundance proteins initially contained in the normal and disease total protein samples.

As used herein, the term “antibody” refers to a protein produced in the body (human or animal) in response to contact with a pathogen or other moiety not recognized as self. Antibodies (such as, for example, IgG and IgY) and antibody fragments (such as, for example, Fab and scFv) have the specific capacity of neutralizing, hence creating immunity to, the pathogen.

As used herein, the term “antigen” refers to any substance capable of inducing a specific immune response and of reacting with the resulting antibodies produced by that response.

Antibodies have been used as cancer research reagents and as diagnostic tools in many different formats. Because of their high specificity and affinity, which can detect targets at picogram (pg) levels, antibodies are used in studying expression, functions, and localization of proteins, steroid hormones, and many other biological molecules. However, because current antibody-based assays are limited to analyzing the presence of known proteins, these assays are insufficient for cancer proteomic analysis because many proteins potentially associated with cancers are almost certainly unknown.

Moreover, current sources of antibodies are mainly from mammalian species such as rabbits, mice, rats, sheep, and goats. Due to the close homology of proteins between humans and these mammalian species, production of antibodies by these animals against similar proteins and peptide antigens is difficult because these proteins may not be recognized as “foreign” upon immunization. Such problems can be reduced by using chickens as hosts for the production of polyclonal antibodies, since proteins from chickens share less homology with human proteins. The nature and magnitude of an antibody response is dependent on the protein structure being foreign or self, the amount of protein being immunized, and the route of immunization. If the amount of a protein is too little, for example, in the case of low-abundance proteins in a biological sample, no antibody will be produced in response to the immunization. The novel strategy provided in the present disclosure provides a method to make chicken antibodies against commonly shared abundance and antigenic proteins (but not low-abundance proteins) and use the antibodies for affinity depletion of these commonly shared abundant proteins.

There are many other advantages to using avian animals as hosts rather than mammalian species. Suitable avian animals include chickens, turkeys, ducks, geese and the like. Preferably, chickens (laying hens) are used as the host animals. First, antibodies can be obtained directly from eggs, minimizing the handling of animals after immunization and eliminating the invasive procedures needed for collecting blood, including sacrificing animals. Second, chickens eggs are rich with antibodies that can be easily purified. One egg can contain about 80 mg of chicken IgY (IgG in mammals). The amount of IgY per egg is equal to the same amount of IgG from about 40 mL of rabbit blood. Third, a large quantity of antibodies can be obtained from chickens. In general, one laying hen can produce about 25 eggs per month, containing about 2000 mg IgY antibodies. Fourth, it is easier to raise and care for chickens than laboratory mammals because there is less need for special equipment and extensive care, and it is a humane way to produce chicken polyclonal antibodies since chickens are not sacrificed in the process. Fifth, chicken and mammalian antibodies have been demonstrated to have similar affinity. Chickens have been shown to be more efficient than mammals in making abundant, cheaper and high-avidity antibodies against mammalian antigens.

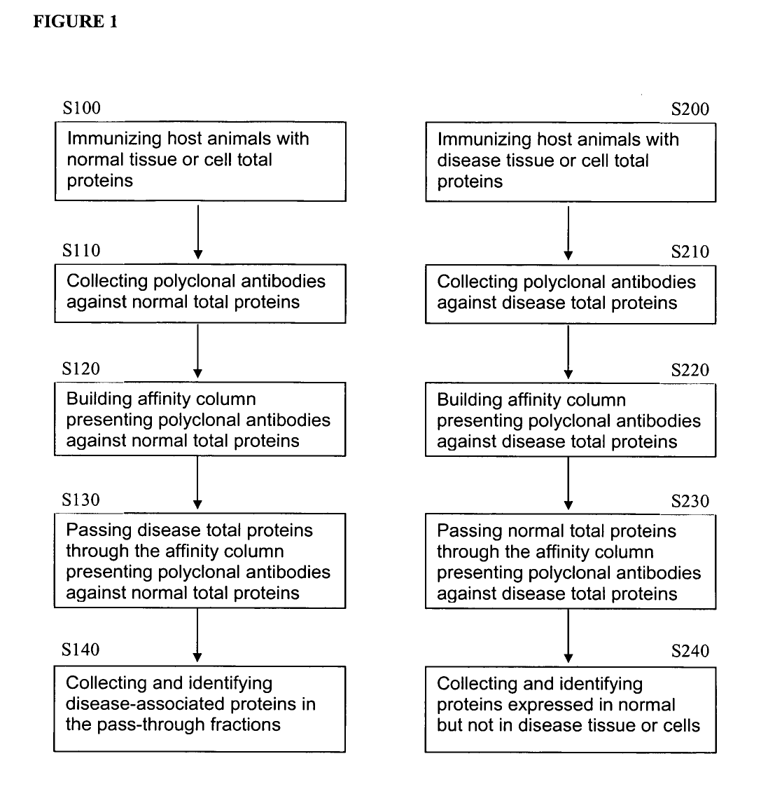

Referring to FIG. 1, a method is provided to remove commonly shared proteins by global polyclonal antibody columns for enrichment of potential protein biomarkers associated with specific disease or biological function. In step S100, host animals, such as hens, are immunized with normal tissue or cell total proteins. In step S110, polyclonal antibodies against normal total proteins are collected. In step S120, affinity column presenting polyclonal antibodies against normal total proteins are built. In step S130, disease total proteins are passed through the affinity column presenting polyclonal antibodies against normal total proteins. Proteins unique to the disease and proteins with low abundance pass through. Common, abundant and antigenic proteins are retained on the column. In step S140, cancer-associated proteins in the pass-through fractions are collected. The pass-through fractions can be enriched and further identified to discover low abundance and unique proteins.

Alternatively, in step S200, host animals, such as hens, are immunized with disease tissue or cell total proteins. In step S210, polyclonal antibodies against disease total proteins are collected. In step S220, affinity column presenting polyclonal antibodies against disease total proteins are built. In step S230, normal tissue or cell total proteins are passed through the affinity column presenting polyclonal antibodies against disease total proteins. Proteins unique to the normal tissue or cell and proteins with low abundance pass through. Common, abundant & antigenic proteins are retained on the column. In step S240, proteins expressed in normal but not in disease tissue or cells are collected. The pass-through fractions can be enriched and further identified to discover low abundance and unique proteins.

Any suitable methods can be used to immunize the host animals with total proteins of a biological sample and to collect polyclonal antibodies against the total proteins from the resultant host animals. For example, polyclonal antibodies can be prepared by dispersing the antigen, such as the total proteins of the biological sample, in a physiologically-tolerable diluent such as saline, to form an aqueous composition. An immunostimulatory amount of the aqueous composition, with or without adjuvant, is administered to a host animal and the inoculated animal is then maintained for a time period sufficient for the antigen to induce anti-antigen antibodies. Boosting doses may be used in individuals that are already primed to respond to the antigen. Antibodies can include antibody preparations from a variety of commonly used animals, e.g., hens, goats, primates, donkeys, swine, rabbits, horses, guinea pigs, rats, and mice, and even human antibodies after appropriate selection, fractionation and purification.

The induced antibodies can be harvested and isolated to the extent desired by well known techniques, such as by alcohol fractionation and column chromatgraphy, or by immunoaffinity chromatography. Representative methods are set forth, for example, in Howard et al. (Ed.), Making and Using Antibodies: A Practical Handbook, CRC Press (2006); Harlow et al., Antibodies: A Laboratory Manual, Cold Spring Harbor Laboratory Press (1988); and Harlow et al., Using Antibodies: A Laboratory Manual, Cold Spring Harbor Laboratory Press (1998), the entireties of all of which are hereby incorporated by reference.

Any suitable methods can be used to prepare an affinity column using the polyclonal antibodies against the total proteins. The polyclonal antibodies can be coated to the affinity column covalently or non-covalently. The polyclonal antibodies also can bind to a support first and the support coated with the polyclonal antibodies can be packed to a column. The support can be beads. Representative methods are set forth, for example, in Hage et al. (Ed.), Handbook of Affinity Chromatography, 2nd Ed., CRC Press (2005); Mallia et al., Immobilized Affinity Ligand Techniques, Academic Press (1992); and Kline (Ed.), Handbook of Affinity Chromatography, Marcel Dekker (1993), the entireties of all of which are hereby incorporated by reference.

The global polyclonal antibodies from chickens produced using the novel method disclosed in the present disclosure are effective against multiple human proteins. In one example, the global polyclonal antibodies removed over about 98% of target proteins (commonly shared proteins) in a total protein sample by an affinity column coated with the global polyclonal antibodies. Specifically, total proteins from either normal or disease tissues were immunized to chickens to generate global polyclonal antibodies. The chicken global polyclonal antibodies against the total proteins were used to build affinity columns. The global polyclonal antibodies affinity columns were used to remove commonly shared proteins from the samples to be studied. After the affinity depletion of commonly shared proteins, unique proteins or potential protein markers were enriched for further analysis.

### EXAMPLE

Enrichment of Differentially Expressed Proteins by Reciprocal Affinity Depletion (RAD)—Rat Lung Proteins

Two laying chickens (hens) were immunized with normal rat lung total proteins. Two other hens were immunized with asthmatic rat lung total proteins at about 1 mg/immunization per hen. The asthmatic phenotype was induced by Sendai virus infection early in life. Four immunizations were conducted. Eggs were collected after the third immunization, and total egg yolk IgY was isolated. Antibodies against the normal lung proteins or the asthmatic lung proteins were affinity purified and were used to set up 3 mL affinity columns, i.e., an anti-normal column and an anti-asthmatic column. The binding capacities of the columns were estimated to be about 3 mg protein per mL beads. The affinity columns were used to reciprocally deplete proteins binding to the antibodies (the “RAD” method). Specifically, in order to deplete commonly shared, abundant antigenic proteins from biological samples, total lung proteins (about 20 mg) from asthmatic rats were passed through the anti-normal column three times, the flow-through proteins were collected. Similarly, about 20 mg of total lung proteins from normal rats were passed through the anti-asthmatic column three times, and the flow-through proteins were collected. The flow-through proteins were concentrated and measured for protein concentration. The final yield of pass-through from the 20 mg original samples was about 1 mg from the normal sample and about 0.9 mg from the asthmatic sample, indicating a depletion of about 95% total proteins.

SDS-PAGE analysis of proteins before and after running through the RAD columns showed there were many protein bands before the affinity depletion of asthmatic lung total proteins and normal lung total proteins. However, most of these bands disappeared after affinity depletion of asthmatic proteins and normal proteins. These abundant proteins were bound to the affinity columns. The SDS-PAGE showed a protein band, with a molecular weight below 18 kDa, that remained in the pass-through sample. This protein could be highly conserved between rat and chicken or non-antigenic, which would lead to an inability of the chicken to produce the specific antibody. The data illustrated that using chicken anti-total protein antibody RAD columns, most, if not all, of the abundance proteins were removed. Since the antibodies are made against all known and unknown shared abundance antigenic proteins, such affinity columns are also referred to as global abundance protein affinity removal (GAPAR) columns.

In order to determine whether there were any unique proteins differentially expressed between asthmatic lung and normal lung, the unbound proteins, as well as the original total protein samples, were analyzed on 2-DE gels. The proteins were run on 2-DE gels (about 100 μg/gel) in duplicate and visualized with Silver Stain (Kendrick Labs Inc., Madison, Wis.). The normal and asthmatic gels were overlaid and manually compared for identification of protein spots that were different based on their unique presence or higher intensity in one gel compared to the other. Multiple protein spots unique to or enriched in the asthmatic lung sample were identified. Similarly, multiple protein spots unique to or enriched in the normal control lung sample were identified. The differences were confirmed on duplicate 2-DE gels. Specifically, after passed through the GAPAR column, many proteins, especially those with high abundance and high molecular weights, disappeared. Only 3 subtle differences could be observed for the original samples, while 14 strong differences were observed for the reciprocal affinity depleted samples. It was estimated that approximately 840 protein spots could be visualized on the original sample whereas only about 338 spots could be visualized after reciprocal affinity depletion. These data illustrated that the GAPAR column removed most, if not all, of the commonly shared, abundant antigenic proteins, and allowed those proteins differentially expressed, as well as those with low abundance and non-antigenicity, to flow through.

Computerized Comparisons of Protein Spots Between Asthmatic and Control Samples after RAD

Duplicate gels were obtained from each RAD sample and were scanned with a laser densitometer (Model PDSI, Molecular Dynamics Inc, Sunnyvale, Calif.). The scanner was checked for linearity prior to scanning with a calibrated Neutral Density Filter Set (Melles Griot, Irvine, Calif.). The images were analyzed using Progenesis PG240 software with TT900 (version 2006, Nonlinear Dynamics). The general method of computerized analysis for these pairs included image warping with TT900 software followed by automatic spot finding, background subtraction (average on border), matching, and quantification in conjunction with detailed manual checking. Spot percentage is equal to spot integrated density (volume) expressed as a percentage of total density of all spots measured. Spot percentages are given to indicate relative abundance. Difference is defined by the fold-change of the spot percentage from a gel compared to the matched spot of the comparison gel. For example, if both spots have the same spot percentage, the difference field will display 1.0. If the spot in the comparison gel has a spot percentage twice as large, the difference field will display 2.0, indicating 2-fold up-regulation. If the spot in the comparison gel has a value half as large, the difference field will display—2.0, indicating 2-fold down-regulation. Student's t test values, generated by the software for an n of 2 gels/sample, were used along with fold difference as criteria for differences.

Referring to Table 1, the computerized comparison of protein spots between RAD asthmatic and RAD control samples on 2-DE gels was summarized. Reference spot numbering, pI, and molecular weights were given for changing polypeptide spots analyzed in the samples of averaged RAD asthmatic lung proteins (gels 2310 #8-9) and averaged RAD control lung proteins (gels 2310 #10-11). Also shown were fold increases or decreases (difference) of the polypeptides for asthmatic lung versus control lung, and P values from Student's t-test to show the significance of the differences. The differences were calculated from spot percentages (individual spot density divided by total density of all measured spots). Spot percentages indicated relative abundance. The p values were for n=2 gels/sample. A total of 338 spots were analyzed.

Still referring to Table 1, a total of 338 spots could be analyzed, and only those spots that were increased in Asthmatic Lung versus Control Lung by more than about 1.7 fold and/or having a P value less than about 0.05, or decreased in Asthmatic Lung versus Control Lung by less than or equal to about −1.7 fold and/or having a p value less than about 0.05 were shown. These spots are considered to be significantly different in expression (density) between the asthmatic and control lung samples. A total of 19 proteins were up-regulated while 21 proteins were down-regulated in asthmatic lung. A computerized comparison of the original total proteins on 2-DE gels did not yield similar results due to the presence of many abundance proteins.

TABLE 1
Computer Analysis of Differential Protein Expression on 2-
DE Gels for Asthmatic Lung And Control Lung after RAD.

| | | | Asthmatic | Asthmatic | | Control | Control | | Asthmatic | Asthmatic |
|---|---|---|---|---|---|---|---|---|---|---|
| | | | 2310 | 2310 | Averaged | 2310 | 2310 | Averaged | Vs | Vs |
| | | | #8 | #9 | Asthmatic | #10 | #11 | Control | Control | Control |
| Spot # | pI | MW | Spot % | Spot % | Spot % | Spot % | Spot % | Spot % | Difference | T-test (p) |
| 12 | 5.6 | 130,003 | 0.050 | 0.046 | 0.048 | 0.018 | 0.018 | 0.018 | 2.7 | 0.004 |
| 15 | 6.2 | 110,290 | 0.023 | 0.027 | 0.025 | 0.002 | 0.005 | 0.004 | 6.9 | 0.009 |
| 19 | 6.0 | 89,366 | 0.054 | 0.054 | 0.054 | 0.024 | 0.030 | 0.027 | 2.0 | 0.013 |
| 45 | 6.9 | 64,057 | 0.357 | 0.424 | 0.390 | 0.180 | 0.138 | 0.159 | 2.5 | 0.028 |
| 47 | 7.2 | 63,697 | 0.133 | 0.269 | 0.201 | 0.046 | 0.051 | 0.049 | 4.1 | 0.268 |
| 68 | 7.1 | 54,795 | 0.063 | 0.106 | 0.084 | 0.015 | 0.021 | 0.018 | 4.8 | 0.090 |
| 69 | 6.9 | 54,619 | 0.018 | 0.025 | 0.021 | 0.007 | 0.006 | 0.006 | 3.4 | 0.055 |
| 70 | 7.3 | 54,090 | 0.096 | 0.173 | 0.135 | 0.041 | 0.043 | 0.042 | 3.2 | 0.251 |
| 71 | 7.5 | 53,560 | 0.176 | 0.264 | 0.220 | 0.084 | 0.095 | 0.089 | 2.5 | 0.099 |
| 72 | 5.2 | 52,910 | 0.006 | 0.006 | 0.006 | 0.002 | 0.002 | 0.002 | 3.1 | 0.014 |
| 82 | 6.8 | 49,855 | 0.148 | 0.152 | 0.150 | 0.075 | 0.094 | 0.085 | 1.8 | 0.022 |
| 115 | 5.7 | 39,762 | 0.173 | 0.133 | 0.153 | 0.039 | 0.041 | 0.040 | 3.8 | 0.029 |
| 151 | 7.2 | 35,524 | 0.347 | 0.460 | 0.404 | 0.076 | 0.083 | 0.080 | 5.1 | 0.029 |
| 167 | 6.6 | 33,320 | 0.047 | 0.048 | 0.047 | 0.022 | 0.030 | 0.026 | 1.8 | 0.030 |
| 189 | 7.8 | 31,162 | 0.061 | 0.078 | 0.070 | 0.017 | 0.020 | 0.018 | 3.8 | 0.026 |
| 190 | 8.2 | 31,162 | 0.260 | 0.318 | 0.289 | 0.063 | 0.076 | 0.070 | 4.1 | 0.017 |
| 258 | 7.6 | 25,457 | 0.520 | 0.671 | 0.595 | 0.126 | 0.159 | 0.142 | 4.2 | 0.028 |
| 260 | 8.0 | 25,364 | 0.895 | 1.176 | 1.035 | 0.133 | 0.196 | 0.165 | 6.3 | 0.026 |
| 268 | 8.4 | 24,402 | 0.300 | 0.339 | 0.320 | 0.187 | 0.177 | 0.182 | 1.8 | 0.020 |
| 4 | 5.5 | 185,712 | 0.004 | 0.005 | 0.004 | 0.011 | 0.019 | 0.015 | −3.4 | 0.109 |
| 6 | 6.5 | 166,856 | 0.485 | 0.332 | 0.408 | 0.862 | 0.921 | 0.891 | −2.2 | 0.028 |
| 8 | 6.1 | 160,857 | 0.020 | 0.015 | 0.018 | 0.032 | 0.038 | 0.035 | −2.0 | 0.050 |
| 10 | 5.8 | 140,287 | 0.109 | 0.100 | 0.105 | 0.271 | 0.237 | 0.254 | −2.4 | 0.014 |
| 14 | 6.0 | 114,575 | 0.097 | 0.083 | 0.090 | 0.183 | 0.208 | 0.195 | −2.2 | 0.018 |
| 17 | 5.5 | 100,005 | 0.160 | 0.103 | 0.132 | 0.271 | 0.288 | 0.280 | −2.1 | 0.038 |
| 23 | 6.9 | 87,298 | 0.018 | 0.012 | 0.015 | 0.068 | 0.050 | 0.059 | −4.0 | 0.041 |
| 48 | 5.4 | 62,781 | 0.061 | 0.052 | 0.056 | 0.305 | 0.351 | 0.328 | −5.8 | 0.007 |
| 57 | 5.1 | 58,701 | 0.009 | 0.005 | 0.007 | 0.016 | 0.015 | 0.016 | −2.2 | 0.049 |
| 84 | 4.1 | 48,642 | 0.178 | 0.173 | 0.176 | 0.389 | 0.417 | 0.403 | −2.3 | 0.004 |
| 94 | 6.2 | 46,559 | 0.078 | 0.086 | 0.082 | 0.157 | 0.169 | 0.163 | −2.0 | 0.009 |
| 95 | 6.0 | 46,474 | 0.042 | 0.032 | 0.037 | 0.123 | 0.096 | 0.110 | −2.9 | 0.038 |
| 99 | 7.6 | 42,880 | 0.013 | 0.014 | 0.014 | 0.025 | 0.026 | 0.026 | −1.9 | 0.007 |
| 101 | 6.1 | 42,631 | 0.411 | 0.344 | 0.377 | 0.752 | 0.810 | 0.781 | −2.1 | 0.012 |
| 116 | 6.1 | 39,762 | 0.029 | 0.023 | 0.026 | 0.074 | 0.187 | 0.130 | −5.0 | 0.205 |
| 215 | 5.5 | 28,426 | 0.018 | 0.024 | 0.021 | 0.181 | 0.202 | 0.192 | −9.0 | 0.004 |
| 251 | 7.0 | 26,104 | 1.026 | 0.719 | 0.872 | 2.084 | 1.832 | 1.958 | −2.2 | 0.032 |
| 261 | 8.3 | 25,302 | 0.194 | 0.168 | 0.181 | 0.311 | 0.304 | 0.308 | −1.7 | 0.011 |
| 272 | 7.3 | 24,466 | 0.240 | 0.417 | 0.329 | 0.758 | 0.788 | 0.773 | −2.4 | 0.038 |
| 278 | 5.6 | 23,170 | 0.068 | 0.111 | 0.089 | 0.671 | 0.518 | 0.594 | −6.7 | 0.024 |
| 283 | 5.6 | 22,279 | 0.047 | 0.032 | 0.040 | 0.254 | 0.211 | 0.233 | −5.9 | 0.013 |

Characterization of Protein Spots Differentially Expressed from RAD Using MALDI-TOF MS

The RAD prepared protein samples (about 600 μg) from either asthmatic lung or normal lung tissues were re-run on 2-DE gels and visualized with Coomassie blue stain. The gels were compared with the corresponding silver stained gels. Similar patterns were observed for all protein spots. Eleven spots from asthmatic lung and two spots chosen from normal lung were cut out from the Coomassie gels for MALDI-TOF MS analysis (Kendrick Labs) to identify the proteins. The eleven protein spots from the asthmatic lung were found to be glutathione S-transferase pi (GSTP1), glutamate dehydrogenase (DHE3), gelsolin, moesin, calreticulin, annexin A1, annexin A1/keratin, annexin A1, vinculin, protein disulfide isomerase, and SEC14-like protein 3. Three different spots were characterized as annexin A1, which could be due to proteolysis of annexin-1 in asthma. In addition, several of these proteins, such as GSPT1 and gelsolin, have been shown to be associated with asthma. The two spots from the normal lung were found to be hemopexin and serotransferrin. These results illustrated that disease-relevant proteins can be identified using the RAD method along with other proteomic technologies such as 2-DE and MALDI-TOF MS.

Depletion of Immunoglobulin Gamma (IgG) Contained in Original Normal Human Plasma Total Proteins Using GAPAR Column. The total IgG concentration in human plasma is about 11 mg/mL and the total protein concentration in human plasma is about 75 mg/mL. Original total plasma proteins (about 20 μg) from a normal individual were passed through the GAPAR column. The lung cancer plasma proteins before and after passing through the GAPAR column were analyzed using western blotting (about 20 μg/lane). Similarly, normal plasma proteins before and after passing through the GAPAR column were analyzed as well (about 20 μg/lane). The results showed that there was no detectable IgG in both the normal plasma and lung cancer plasma samples after passed through the GAPAR column, suggesting abundance proteins in the plasma, such IgG, have been depleted by the GAPAR column.

Affinity Depletion of Commonly Shared Abundance Proteins in Lung Cancer And Normal Plasma with Biotinylated Anti-Normal Plasma Total Protein IgY Beads. Original total plasma proteins from a lung cancer patient were passed through biotinylated anti-normal plasma IgY global polyclonal antibodies and streptavidin beads. The lung cancer plasma proteins after depletion of abundance proteins with the biotinylated anti-normal plasma IgY global polyclonal antibodies and streptavidin beads were collected. The original total plasma proteins from a lung cancer patient before and after affinity depletion and the original total plasma proteins from a age- and sex-matched normal individual were analyzed using SDS-PAGE. The results showed that most, if not all, of abundance proteins, such as albumin, were depleted after affinity depletion by the biotinylated chicken global polyclonal anti-plasma antibodies and streptavidin beads.

Experimental

To generate global polyclonal antibodies for setting up columns for reciprocal affinity depletion of abundance proteins shared between samples, such as between normal and asthmatic lung samples, chickens were used as hosts for making the global polyclonal antibodies because, compared with mammalian hosts, their proteins have significantly less homology with human counterparts. Other animals can be used if their proteins also have significantly less homology with the species from which the samples are derived. In the experiments disclosed below, an inbred line of laying Leghorn chickens was used. Leghorn is the major breed of chickens used for consumer egg production in the United States due to its high laying capacity, about 25 eggs per month. Leghorn chickens make highly homogeneous polyclonal antibodies. Both human plasma samples and rat lung total protein samples were used in the experiments.

Sample Preparation

Lung tissues were obtained from both normal and Sendai virus-induced asthmatic rats (Medical School of University of Wisconsin-Madison). Lung total proteins were prepared using T-PER solution from Pierce Biotechnology (Rockford, Ill.) according to the manufacturer's instructions. Lung total proteins were dialyzed against PBS before use. Human plasma samples were obtained from both normal individuals and lung cancer patients and were diluted in PBS accordingly in the experiments. Protein concentrations of both lung protein extracts and human plasma samples were measured using a BCA kit (Pierce Biotechnology).

Generation of Global Polyclonal Antibodies By Immunizing Chickens with Total Proteins

To generate chicken global polyclonal antibodies, laying hens were immunized with about 1-4 mg total proteins per immunization: about 1 mg per immunization for lung proteins, and about 4 mg per immunization for human plasma total proteins. Four laying hens were used for each protein sample. Prior to the immunizations, protein samples (adjusted to about 0.5 mL each in sterile PBS) were mixed and emulsified with an equal volume of adjuvant (complete adjuvant for the first immunization and incomplete adjuvant thereafter). Each immunization was done with multiple injection sites (about 4-5 sites per hen), subcutaneously for the first immunization and intramuscularly for the second and later immunizations. A total of four immunizations per protein sample were done, with an interval of two weeks between immunizations; i.e., at day 0, day 14, day 28, and day 42. Eggs were collected daily after the second immunization until day 72, and crude IgY was prepared from the egg yolks of each hen using GeneTel's standard IgY isolation protocol. Crude IgY was pooled from eggs collected from day 21 to day 28, day 29 to day 42, and day 43 to day 72 from each chicken, corresponding to collection periods after the second immunization, the third immunization, and the fourth immunization, respectively. Protein concentrations of crude IgY were measured using a spectrophotometer based on its OD280 divided by 1.4. The IgY was analyzed for its antibody response using direct ELISA, i.e., an ELISA plate coated with about 2 μg/mL respective total proteins were incubated with crude IgY (about 1000 ng/mL to about 15.625 ng/mL titrations). The binding of IgY to the coated proteins was detected with Rabbit Anti-IgY HRP conjugate (GeneTel). The IgY samples with the highest titer from each protein sample were pooled and used for affinity purification of global polyclonal antibodies.

Affinity Purification of Chicken Global Polyclonal Antibodies And Establishment of Global Polyclonal Antibody Affinity Columns for RAD

Crude IgY from eggs contained about 5-10% specific antibodies when the chickens were immunized with total proteins. If the crude IgY is used to build affinity columns for immuno-depletion, such a column will have a large volume. Since a sample passed through a column will be diluted largely based on the size of the column, protein samples after passing through a crude IgY affinity column will be heavily diluted. Laborious concentration steps will be needed to concentrate the unbound proteins. Therefore, purification of global polyclonal antibodies is needed to build a smaller affinity column for easier handling of protein samples.

Total protein columns were built for affinity purification of global antibodies against the proteins that were used to immunize laying hens. Total proteins from normal or asthmatic lungs, or from human plasma, were chemically cross-linked to activated beads (cyanogens bromide-activated beads from Pierce Biotechnology, Rockford, Ill.) via the amino groups. The capacity of the activated beads was about 15 mg protein per mL. About 30 mg of the total proteins from each sample was used to build 5-mL columns. Crude IgY from hens immunized with the total proteins of each sample was passed through its respective protein column to obtain specific global polyclonal antibodies. After each column was completely washed with PBS, the chicken global polyclonal antibodies were eluted with elution buffer (citrate buffer, pH about 3.0) and immediately neutralized to pH about 7 with 1M Tris buffer (pH 8.0). The eluted global antibodies were dialyzed overnight with three changes of PBS.

Affinity pure global antibodies (from about 150 mg to about 200 mg) against total proteins of normal rat lung, asthmatic rat lung, normal human plasma and human plasma from a lung cancer patient were obtained. Each of the affinity-purified global antibodies was cross-linked to about 5 mL cyanogen bromide-activated beads (Pierce) to build the four different global polyclonal antibody columns. About 1 mL beads for each affinity pure global antibody were used to determine protein binding capacity and the rest of the about 4 mL beads were used for RAD studies.

Determine The Binding Capacity of The Global Polyclonal Antibody Columns

Excess total proteins (about 20 mg each) from each protein sample were run over the respective 1 mL column at 4° C. After washing out the unbound proteins with PBS, the column bound proteins were eluted, and were measured for its protein concentration. The total amount of eluted proteins was the binding capacity of the 1 mL column. The binding capacity of the four global polyclonal antibody columns was about 3-5 mg per mL beads.

Reciprocal Affinity Depletion (RAD)

To remove commonly shared abundant and antigenic proteins and to enrich low abundance, differentially expressed non-antigenic proteins, about 20 mg of total proteins from each normal control were passed through the 4 mL anti-disease column while about 20 mg of disease total proteins were passed through the 4 mL anti-control column. The pass-through (unbound proteins) fractions were collected and measured for protein concentrations based on OD280. Fractions that had larger than about 0.050 OD280 were pooled and concentrated using Zeda Desalt Protein Spin Columns (Pierce). The columns were washed thoroughly with PBS, and the column-bound proteins were eluted with citrate elution buffer (pH about 3). All of above steps were performed in a cold room (about 4° C.). Protein concentrations were determined using the Pierce BCA Protein Assay Kit. If the collected total unbound proteins were more than about 6% of its original total proteins, the unbound proteins were passed through the column again after the column was eluted and washed. Here the total proteins were run three times on the RAD column to obtain the unbound proteins with an amount of about 5% of its original total proteins. Using this depletion approach, low abundance proteins that would contain proteins that were uniquely or differentially expressed between the normal controls and the disease samples were obtained.

Estimating the Effectiveness of Using Chickens to Produce Global Polyclonal Antibodies

Chickens have been shown to be highly effective in generating a strong antibody response against mammalian proteins when injected at the μg to mg levels. In order to use chicken antibodies against abundant and antigenic proteins, which do not recognize low abundance proteins, to set up affinity columns for the removal of abundance proteins, the minimal level or the threshold of each protein in a biological sample that can be removed using the anti-total protein antibody column has to be determined. In other words, those commonly shared abundance proteins in the injected total proteins have to be immunogenic (antigenic) in order to elicit an antibody response in chickens while those low abundance proteins have to be non-immunogenic. To elicit an antibody response, any injected protein should present in an adequate amount and should be antigenic to the chicken, otherwise it will induce immuno-tolerance.

To test for the efficacy of a specific antibody response in chickens, normal human plasma (4 mg protein per immunization) were spiked with two recombinant proteins, human Rab4 (molecular weight: 38 kDa) and mouse NGF (2.5S, molecular weight: 12.7 kDa) at various amounts (from about 0.5 ng to about 5 μg) for immunizations of chickens. The antigenicity (epitopes) of these two proteins was analyzed using DNAStar software (DNAStar, Madison, Wis.). Two chickens per treatment were immunized for four times with the respective proteins. The immunization interval was two weeks apart. Eggs were collected prior to the first immunization, and after the second and later immunizations. Crude IgY were isolated from egg yolks. Antibody responses were measured using a direct ELISA method in which ELISA plates were coated with about 2 μg/mL of the respective proteins. Crude IgY was titrated from about 8 μg/mL down to about 0.125 μg/mL and incubated on the ELISA plate for about 2 hours. Anti-IgY HRP conjugate (RCYHRP, GeneTel) was incubated with the plate after washing. HRP substrate was added after washing for color development. Two minutes after adding the substrate for color development, the reaction was stopped with about 1M HCl.

The data demonstrated that, while the antibody responses to the plasma total proteins were strong after the second immunization, there was no antibody response against NGF at less than about 50 ng NGF per immunization even after the fourth immunization. Anti-NGF antibody response was only observed with more than about 500 ng NGF per immunization after the third immunization. For Rab4, a weak antibody response could be observed with about 5 ng protein per immunization after the third immunization, and a stronger antibody response could be observed with more than about 50 ng Rab4 per immunization. Therefore, using more Rab4 per immunization resulted in a stronger antibody response. Epitope analysis using DNAStar Protean software showed that NGF has 4-5 antigenic sites (epitopes) while Rab4 has about 11 epitopes. This would suggest that the antigenicity of Rab4 is stronger than that of NGF and might explain why more NGF is needed for eliciting the antibody responses. The data demonstrated that chickens do not respond to less than about 5 ng protein that has a high antigenic property. For those proteins with low antigenicity, much more protein per immunization will be needed (more than about 500 ng) to elicit an antibody response. Therefore, the antibody response in the chicken is highly dependent on the structure (antigenicity) and the amount of each protein being immunized.

Determination of RAD Column Depletion Efficacy By SDS-PAGE Gel Analysis

To determine if the commonly shared abundant proteins were depleted, the column-unbound proteins and the eluted column-bound proteins along with the original total proteins were run on a SDS-PAGE 1-dimensional gel (1-DE) at about 30 μg/lane of the column-unbound proteins, and about 80 μg/lane of the original or column-bound proteins. From the 1-DE gel SDS-PAGE analysis, abundant protein depletion was observed. However, due to its limitations in protein separation, the 1-DE gel did not allow direct individual comparison of proteins among the RAD samples. Thus, a better separation technique, 2-DE gel analysis as described below, were used to analyze potential proteomic differences between RAD samples from the disease protein samples versus control protein samples.

Analysis of Unbound Protein Samples (RAD Samples) by 2-DE Gel And MALDI-TOF MS.

2-DE gel and MALDI-TOF MS were used to analyze the presences of proteins differentially expressed between the disease and control samples to validate the feasibility of the global antibodies column for the depletion of commonly shared abundant proteins. After the depletion of commonly shared abundant proteins, the RAD (column-unbound) samples were run in duplicate on 2-DE gels along with the respective original total protein samples, with about 100 μg proteins per gel. Proteins from the 2-DE gels were silver stained for visualization. The RAD sample from normal control rat lung was compared with the asthmatic lung RAD sample. Duplicate gels obtained from each RAD sample were scanned with a laser densitometer (Model PDSI, Molecular Dynamics Inc, Sunnyvale, Calif.). The images were analyzed using Progenesis PG240 software with TT900 (version 2006, Nonlinear Dynamics). The general method of computerized analysis included image warping with TT900 software followed by automatic spot finding, background subtraction (average on border), matching, and quantification in conjunction with detailed manual checking. Spot percentage (the spot integrated density expressed as a percentage of total density of all spots measured) was calculated. Spot percentage was given to indicate relative abundance. Differences were defined by the fold-change of the spot percentage from a gel compared to the matched spot of the comparison gel. For example, if both spots had the same spot percentage, the difference field would display 1.0. If the spot in the comparison gel had a spot percentage twice as large, the difference field would display 2.0, indicating a 2-fold up-regulation. If the spot in the comparison gel had a value half as large, the difference field would display—2.0, indicating a 2-fold down-regulation. Student's t test values were generated by the software for an n of 2 gels per sample. Only those spots that increased or decreased by more than about 1.7 fold and/or had a p value less than about 0.05 were considered to have a significant difference in protein expression.

After protein spots were observed differentially on the 2-DE silver stained gels between the control lung and asthmatic lung samples, 800 μg of each of the RAD samples were run on 2-DE gels. The gels were stained with Coomassie blue. Unique protein spots were excised and subjected to MALDI-TOF MS analysis for protein identification.

Many modifications and other embodiments of the present disclosure will come to mind to one skilled in the art to which the present disclosure pertains having the benefit of the teachings presented in the foregoing description; and it will be apparent to those skilled in the art that variations and modifications of the present disclosure can be made without departing from the scope or spirit of the present disclosure. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A method for removing abundance proteins from a biological sample, the method comprising:
passing the biological sample through a support, the support being coated with an avian polyclonal antibody, the avian polyclonal antibody capable of binding to substantially all proteins in the biological sample with concentrations higher than a predetermined value; and
collecting the pass-through fractions of the biological sample.

2. The method of claim 1, wherein the avian polyclonal antibody is a chicken polyclonal antibody.

3. The method of claim 1, wherein more than about 95% of the proteins in the biological sample bind to the support coated with the avian polyclonal antibody.

4. A method for enriching a protein associated with a certain disease, the method comprising:
removing proteins from a disease biological sample using an avian polyclonal antibody against total proteins of a corresponding normal biological sample; and
collecting the pass-through fractions of the disease biological sample.

5. The method of claim 4, further comprising identifying one or more remaining proteins in the pass-through fractions of the disease biological sample.

6. The method of claim 4, wherein the avian polyclonal antibody is capable of binding to substantially all abundance proteins in the disease biological sample.

7. A method for enriching a protein expressed in a normal tissue or cell but not in a corresponding disease tissue or cell, the method comprising:
removing proteins from a normal biological sample using an avian polyclonal antibody against total proteins of a corresponding disease biological sample; and
collecting the pass-through fractions of the normal biological sample.

8. An avian polyclonal antibody against abundance proteins in a biological sample produced by:
immunizing an avian animal with the total proteins of a biological sample; and
collecting the avian polyclonal antibody from the resultant avian animal, the avian polyclonal antibody capable of binding to substantially all proteins in the biological sample with concentrations higher than a predetermined value.

9. The avian polyclonal antibody of claim 8, wherein the predetermined value is about 1 μg/mL.

10. The avian polyclonal antibody of claim 8, wherein the biological sample is selected from a group consisting of body fluid, cells, and tissues.

11. The avian polyclonal antibody of claim 8, wherein the avian animal is egg-laying chicken.

12. The avian polyclonal antibody of claim 11, wherein the collecting comprising collecting eggs from the immunized chicken and separating the avian polyclonal antibody from the egg yolk.

13. The avian polyclonal antibody of claim 8, wherein the avian polyclonal antibody comprises IgY.

14. The avian polyclonal antibody of claim 8, wherein the biological sample is obtained from an individual in a normal state.

15. The avian polyclonal antibody of claim 8, wherein the biological sample is obtained from an individual in a disease state.

16. A device for removing proteins from a biological sample, the device comprising:
a support; and
the avian polyclonal antibody of claim 8 coated to the support.

17. The device of claim 16, wherein the support is a plurality of beads.

18. The device of claim 16, which is a column.

19. The device of claim 16, wherein the avian polyclonal antibody is covalently bound to the support.

20. The device of claim 16, wherein the avian polyclonal antibody is non-covalently bound to the support.
